(19) United States
(12) Patent Application Publication
(10) Pub. No.: US 2003/0051257 A1
(43) Pub. Date: Mar. 13, 2003

(54) Transgenic goat producing milk containing human granulocyte-colony stimulating factor

(76) Inventors: Seung-Won Jin, Kyungki-do (KR); Doo-Soo Lee, Chungcheongnam-do (KR); Tae-Hun Song, Daejeon (KR); In-Young Choi, Kyungki-do (KR); Ook-Joon Yoo, Kyungki-do (KR); Jung-Ho Ko, Daejeon (KR); Ja-Shin Koo, Daejeon (KR); Sang-Tae Shin, Daejeon (KR); Chul-Sang Lee, Daejeon (KR); Nan-Zhu Fang, Daejeon (KR); Deog-Bon Koo, Daejeon (KR); Keon-Bong Oh, Chungcheongbuk-do (KR); Jung-Sun Park, Daejeon (KR); Woo-Sik Youn, Kyungki-do (KR); Guo-Dong Zheng, Daejeon (KR); Sun-Jung Kim, Daejeon (KR); Yong-Mahn Han, Daejeon (KR); Kyung-Kwang Lee, Daejeon (KR)

Correspondence Address: David A Einhorn, Anderson Kill & Olick, 1251 Avenue of the Americas, New York, NY, 10020, US

(21) Appl. No.: 10/182,433

(22) PCT Filed: Jan. 26, 2001
(86) PCT No.: PCT/KR01/00111

(30) Foreign Application Priority Data
Jan. 24, 2001 (KR) 2000-3187

(51) Int. Cl. G01N 33/00

(52) U.S. Cl. 800/3

(57) ABSTRACT

A transgenic goat zygote is developed from a goat zygote comprising a nucleic acid construct containing a nucleotide sequence of a goat β-casein promoter and a nucleotide sequence encoding hG-CSF, which produces milk containing a high concentration of biologically active hG-CSF.

### FIELD OF THE INVENTION

The present invention relates to a goat zygote comprising a nucleic acid construct which expresses human granulocyte-colony stimulating factor(hG-CSF) gene in a mammary gland tissue-specific manner; and a transgenic goat producing milk containing hG-CSF.

### BACKGROUND OF THE INVENTION

hG-CSF is a biologically active glycoprotein whose expression is triggered by an external stimulus, e.g., a bacterial infection or cancer therapy, to stimulate growth and differentiation of hemopoietic stem cells, e.g., granulocytes or macrophages, while its concentration in the host's blood is infinitesimal when the host is healthy.

Since it is not feasible to obtain hG-CSF from human body, attempts have been made to prepare hG-CSF using E. coli or animal cells. The hG-CSF produced using E. coli has fluctuating in vivo activities and unverified safety. Further, the hG-CSF production process using E. coli is uneconomical due to the requirement of expensive equipments and complicated purification procedures, and so is the hG-CSF production process using animal cells.

Therefore, there has existed a need to develop a method for producing biologically active hG-CSF economically. Recently, there have been reported successful attempts to produce biologically active proteins, lactoperin and collagen(U.S. Pat. Nos. 5,633,076, 5,849,992 and 5,895,833), using a transgenic bovine, goat or porcine as a bioreactor. The proteins produced by this method are identical with the corresponding wild-types produced in human body, while the production cost thereof is lower by a factor of 1,000 to 2,000 than the process using E. coli or an animal cell. WO 97/19589 discloses a method for developing a transgenic dwarf goat but the actual production of biologically active proteins has not been demonstrated.

The present inventors have endeavored to develop a method for producing hG-CSF using a transgenic goat by way of using the mammary gland tissue-specific expression system disclosed by the present inventors in Korean Patent Application No. 97-9601 (Korean Patent Application Laid-Open No. 98-73991).

### SUMMARY OF THE INVENTION

Accordingly, it is an object of the present invention to provide a goat zygote comprising a nucleic acid construct which expresses hG-CSF gene in a mammary gland tissue-specific manner.

Further objects of the present invention include:

a method for preparing said goat zygote;

a method for extracting an intact zygote from a goat to which the nucleic acid construct is to be introduced;

a transgenic goat producing milk containing hG-CSF;

a method for preparing the transgenic goat from the goat zygote;

a method for producing hG-CSF using the transgenic goat;

a milk composition containing hG-CSF produced from the transgenic goat; and

a pharmaceutical composition comprising said hG-CSF so produced.

In accordance with one aspect of the present invention, there is provided a goat zygote which comprises a nucleic acid construct containing a nucleotide sequence of a goat β-casein promoter and a nucleic acid sequence encoding hG-CSF.

Other aspects of the present invention encompass:

a method for preparing the goat zygote which comprises microinjecting the nucleic acid construct into an intact goat zygote;

a method for preparing an intact goat zygote which comprises synchronizing a female goat, superovulating the female goat, mating the superovulated female goat with a male goat and recovering a zygote from the mated female goat, characterized in that the synchronizing step is conducted by administering norgestomet and estradiol to the female goat, inserting an implant containing norgestomet to the female goat and removing the implant; and the superovulating step is conducted by administering to the female goat, sequentially at predetermined time intervals, a combined dose of pregnant mare serum gonadotropin (PMSG) and follicle stimulating hormone (FSH), divided doses of FSH, and a combined dose of FSH and human chorionic gonadotropin (hCG);

a transgenic goat developed from the goat zygote which produces milk containing hG-CSF;

a process for preparing the transgenic goat which comprises transferring the goat zygote into a female goat and allowing the goat zygote to develop to term;

a method for producing hG-CSF which comprises producing milk from the transgenic goat and recovering the hG-CSF from the milk;

a milk composition comprising hG-CSF which is produced from the transgenic goat;

hG-CSF which is produced from the transgenic goat; and

a pharmaceutical composition which comprises the hG-CSF and a pharmaceutically acceptable carrier.

### BRIEF DESCRIPTION OF THE DRAWINGS

The above objects and features of the present invention will become apparent from the following description of preferred embodiments taken in conjunction with the accompanying drawings, in which:

FIG. 1 is a schematic diagram showing the schedule for generating synchronization and superovulation of goat;

FIG. 2 is the polymerase chain reaction (PCR) results showing the introduction of the expression cassette in the transgenic goat's genome DNA;

FIG. 3 is the southern blot analysis results showing the introduction of the expression cassette in the transgenic goat's genome DNA;

FIG. 4 is the western blot analysis results showing expression of hG-CSF in the transgenic goat's milk-serum; and

FIG. 5 is a graph showing proliferation of HL-60 cells induced by the transgenic goat's milk-serum.

### DETAILED DESCRIPTION OF THE INVENTION

The nucleic acid construct used in the preparation of the transgenic goat zygote of the present invention contains the nucleotide sequence of a goat β-casein promoter and the nucleotide sequence of an hG-CSF gene. The expression of hG-CSF is controlled by the goat β-casein promoter which is activated specifically in a mammary gland tissue. The hG-CSF gene and goat β-casein promoter are disclosed in GenBank as accession nos. X03656 and M90559, respectively, and can be obtained from human and goat tissues, respectively, or synthesized using a conventional DNA synthesis method. The nucleic acid construct may be prepared according to a conventional method(Sambrook, J. et al., Molecular cloning: a laboratory manual, 2nd ed. Cold Spring Harbor Laboratory Press, New York (1989)). In addition to the goat β-casein promoter and hG-CSF gene, the nucleic acid construct may further comprise a transcription termination region. An exemplary nucleic acid construct is expression cassette pGbc-hGCSF of SEQ ID NO: 1, wherein the goat β-casein promoter has the nucleotide sequence ranging from the goat β-casein promoter to the nucleotide immediately before the translation initiation codon of the exon I of the goat β-casein gene and it controls the expression of the hG-CSF gene located downstream thereof.

The inventive transgenic goat zygote may be prepared by microinjecting the nucleic acid construct into an intact 1-cell stage zygote of a goat. The microinjection may be conducted under an inverted microscope equipped with an micromanipulator according to a conventional method(Manipulating the mouse embryo: A laboratory manual, 2nd ed. Cold Spring Harbor Laboratory Press, New York, (1994)). An example of the inventive zygote is Capra hircus aegagrus embryos/pGbc-bGCSF, which is derived from Capra hircus aegagrus, a species indigenous to Korea, and comprises expression cassette pGbc-bGCSF. This transgenic zygote is deposited on Dec. 28, 1999 with the Korean Collection for Type Cultures (KCTC) (Address: Korea Research Institute of Bioscience and Biotechnology (KRIBB), #52, Oun-dong, Yusong-ku, Taejon, 305-333, Republic of Korea) under the accession number, KCTC 0718BP, in accordance with the terms of Budapest Treaty on the International Recognition of the Deposit of Microorganism for the Purpose of Patent Procedure. However, this zygote does not limit the transgenic goat zygote of the present invention.

The 1-cell stage zygote to be used in the microinjection procedure is prepared by synchronizing a female goat, superovulating the female goat, mating the superovulated female goat with a male goat and recovering zygotes from the mated female goat.

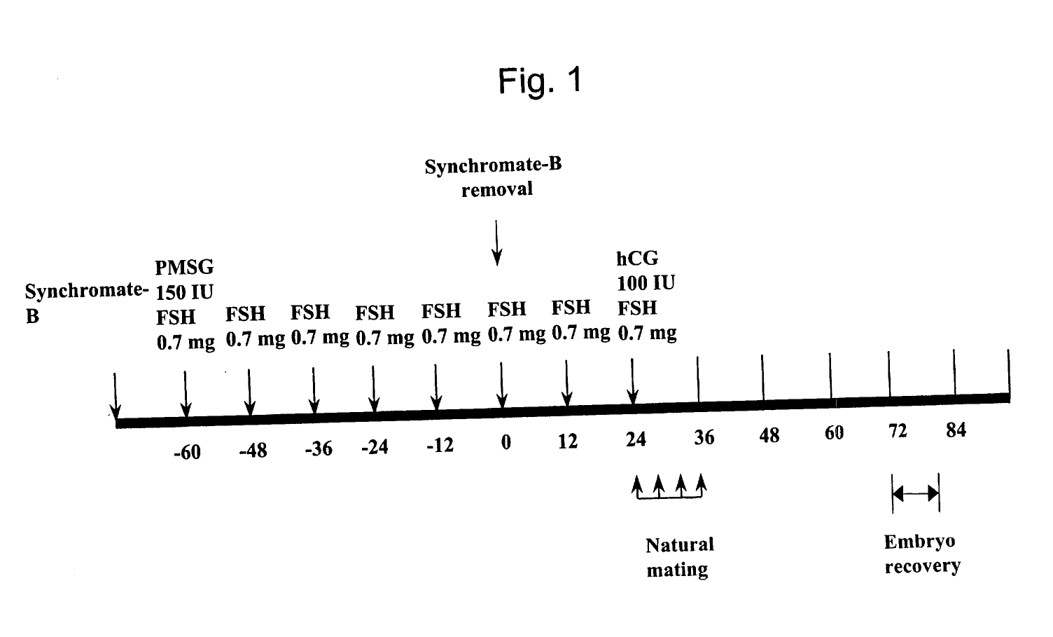

Preferably, the synchronizing and superovulating steps may be conducted according to the schedule shown in FIG. 1: The synchronizing step is conducted by injecting intramuscularly an appropriate amount of norgestomet and estradiol as well as inserting an implant containing an appropriate amount of norgestomet into the ear and removing the implant at day 13 or 14 after the insertion of the implant; and the superovulating step is conducted by injecting PMSG, FSH and hCG eight times every 12 hours starting from 60 hours before the removal of the implant. The injection schedule involves administering PMSG and FSH at the first injection; FSH at the second to the seventh; and FSH and hCG at eighth. The hCG injected together with FSH at the eighth injection is effective in inducing enhanced superovulation as well as in regulating the ovulation time. This method is particularly suitable in preparing an intact zygotes of Capra hircus aegagrus.

The mating and recovering step may be conducted by a conventional procedure. After mating the superovulated female goat with a male goat, the recovering may be conducted by: anesthetizing the mated female by injecting an anesthetic agent, e.g., xylazine or lidocaine, at 72 to 76 hours after the removal of the implant, the mated female goat being fasted for 24 hours prior to the injection; positioning it on its back; locally anesthetizing the abdominal median line; cutting the abdominal median line to remove the ovary, oviduct and uterus; inserting a catheter into the oviductal infundibulum; introducing a phosphate-buffered saline(PBS) containing fetal bovine serum into the catheter to flow from the uterus to the oviduct; and obtaining an intact zygote.

The transgenic goat zygote of the present invention may be transplanted into a female goat(recipient) and allowed to develop to term according to a conventional method. The transplantation may be conducted by fasting a recipient goat for 24 hours; cutting the abdominal median line of the recipient goat to remove the ovary, oviduct and uterus; inserting a catheter carrying the transgenic zygotes into the oviductal infundibulum so that the zygotes can be transferred to the oviduct. The recipient goat which may be used in the present invention is selected from those being in estrus, spontaneous or induced by a hormone, e.g., PMSG. A recipient in the spontaneous estrus mode is preferred. The number of transgenic zygotes that may be transplanted ranges from 2 to 4, per recipient goat. Pregnancy of the recipient goat may be identified with an ultrasonic diagnostic equipment at about day 40 after the transplantation. The transplanted zygotes are allowed to develop to term to obtain transgenic goats whose somatic and germ cells comprise the nucleic acid construct, and then the transgenic goats are bred.

The presence of the inventive nucleic acid construct in the transgenic goat may be identified by a conventional method, e.g., polymerase chain reaction(PCR) or southern blot analysis. Further, the expression of hG-CSF in the transgenic goat may be identified by a conventional method, e.g., western blotting analysis or enzyme-linked immunosorbent assay (ELISA), using its milk proteins.

After maturing, the transgenic goat produces hG-CSF specifically in the mammary gland tissue and release the hG-CSF in the milk. The hG-CSF thus produced exhibits good biological activity maintenance in vivo, and stimulates the growth and differentiation of granulocytes and macrophages. It is well known in the art that hG-CSF is effective in preventing and treating various diseases, e.g., leucopenia caused by bone marrow transplantation, malignant lymphoma, acute leukemia, lung cancer, ovarian cancer, testicular tumor, myelodysplasia, aplastic anemia and congenital neutropenia. Therefore, the hG-CSF of the present invention may be advantageously used in a pharmaceutical composition.

The pharmaceutical formulation may be prepared in accordance with any of the conventional procedures. In preparing the formulation, the active ingredient is preferably admixed or diluted with a carrier, or enclosed within a carrier which may be in the form of a capsule, sachet or other container. When the carrier serves as a diluent, it may be a solid, semi-solid or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, and diluents are lactose, dextrose, sucrose, sorbitol, mannitol, starches, gum acacia, alginates, gelatin, calcium phosphate, calcium silicate, cellulose, methyl cellulose, microcrystalline cellulose, polyvinylpyrrolidone, water, methylhydroxybenzoates, propylhydroxybenzoates, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a mammal by employing any of the procedures well known in the art.

The pharmaceutical composition of the present invention can be administered via various routes including oral, transdermal, subcutaneous, intravenous and intramuscular introduction. In case of human, a typical daily dose of the hG-CSF may range from about 75 to 600 mg/kg body weight, preferably 100 to 400 mg/kg body weight, and can be administered in a single dose or in divided doses.

However, it should be understood that the amount of the active ingredient actually administered ought to be determined in light of various relevant factors including the condition to be treated, the chosen route of administration, the age, sex and body weight of the individual patient, and the severity of the patient's symptom; and, therefore, the above dose should not be intended to limit the scope of the invention in any way.

The following Examples are intended to further illustrate the present invention without limiting its scope.

### EXAMPLE 1

### Construction of Vector pGbc-hGCSF

Plasmid pGbc_S containing the portion of the O-casein gene ranging from the promoter to the exon I derived from Korean native goat(Capra hircus aegagrus)(Korean Patent Application Laid-Open No. 99-73991) was cleaved with HindIII and the resulting mixture was extracted with a 1:1 (v/v) mixture of phenol and chloroform, precipitated in 95% ethanol and dissolved in distilled water to obtain a DNA fragment containing the portion of goat β-casein gene ranging from the promoter to the exon I. The DNA fragment was cleaved with DraI and the resulting mixture was electrophoresed on 1% agarose gel. The band of 1239 bp fragment was cut from the agarose gel and subjected to purification using Geneclean II kit(Bio101, USA) to obtain DNA fragment 1.

Genome DNA of goat (Capra hircus aegagrus) was subjected to PCR using primers CAS-F1 (SEQ ID NO: 2) and CAS-R1 (SEQ ID NO:3) and the PCR product was cleaved with DraI and HindIII and extracted electronically to obtain DNA fragment 2.

DNA fragments 1 and 2 were ligated with opened pBluescript II (Stratagene, USA) obtained by treating with SalI, Hind III and calf alkaline phosphatase. Into the HindIII and EcoRI sites of the resulting plasmid, DNA fragment pRC/RSV containing hG-CSF gene followed by transcription termination region of bovine growth hormone(Invitrogen, Netherlands) was inserted to obtain vector pGbc-hGCSF.

Vector pGbc-hGCSF was cleaved with BssHII and KpnI, and the resulting mixture was electrophoresed on agarose gel, purified using Geneclean II kit (BIO101) and Elutip-d (Schleicher and Schuell, Germany) in sequence, dialyzed against a dialysis solution (10 mM Tris-Cl (pH 7.2) and 0.1 mM EDTA) and then filtered using a 0.22 μm filter (Nalgene, USA) to obtain expression cassette pGbc-hGCSF. The expression cassette thus obtained was diluted to a final concentration of 4 μg/ml with the dialysis solution.

### EXAMPLE 2

### Recovery of Goat Zygote

According to the schedule shown in FIG. 1, 1 to 3-year-old female, Korean native goats (Capra hircus aegagrus), which were provided by Konju Sabisung stock farm, were injected intramuscularly with 2 ml of sesame oil containing 3.0 mg of norgestomet and 5.0 mg of estradiol. After removing the ear hairs and disinfecting the ear, implant Syncromate-B (Sanofi Animal Health, USA) was inserted into the disinfected ear using Syncromate-B gun (PETS, USA)) and then removed surgically at day 13 or 14 after the insertion to synchronize the estrus.

5.6 mg of FSH (Ovagen, Immuno-Chemical Products, New Zealand) was divided into eight doses and injected intramuscularly to the goat every 12 hours from 60 hours, as shown in FIG. 1: 0.7 mg of FSH and 0.7 mg of PMSG (Pregnecol, Horizon Technology, Australia) were injected first, 0.7 mg each of FSH, thereafter until the seventh injection, and 0.7 mg of FSH together with 100 IU of hCG, at the eighth injection. The ovulated female goat was mated with a male goat (Capra hircus aegagrus) for 12 hours.

At 72 to 76 hours after the removal of the implant, 2% xylazine solution (Rompun, Bayer, Korea) was injected intramuscularly to the female goat which had been fasted for 24 hours prior to the injection and the female goat was positioned on its back. 10 ml of 2% lidocaine was injected to the abdominal median line to anesthetize locally and the abdominal median line was cut in a length of 4 to 6 cm to remove the ovary, oviduct and uterus. Catheter, a polyethylene tube having an inside diameter of 1.0 mm, was inserted into the oviductal infundibulum to fix therein and phosphate-buffered saline(PBS) containing fetal bovine serum is introduced into the catheter to flow in the reverse orientation, from uterus to the oviduct, to recover intact zygotes. The intact zygotes were stored in modified synthetic oviductal fluid (m-SOF: Takahashi Y. et al., Theriogenology, 37, 963-978 (1991)) until the following microinjection procedure.

### TEST EXAMPLE 1

### Effect of FSH and hCG on the Ovulation and Zygote Recovery Time

(1) Influence of Combining hCG with FSH on the Ovulation

To examine the influence of combining hCG with FSH on the ovulation of Korean native goat, the procedure of Example 2 was repeated together with a control run which was conducted exactly the same way except that 0.7 mg of FSH was used alone without hCG at the eighth injection. At 70 to 76 hours after the removal of the implant, ovulation rate (number ratio of the ovulated goat to the total goat), ovulation point (average number of the ovulated follicles per ovulated goat), recovery rate (number ratio of the recovered oocyte to the ovulated follicle) and fertilization rate (number ratio of the zygote having pronuclei to the recovered oocyte) were determined.

Results are shown in Table I.

TABLE I
Influence of combining hCG with FSH on the ovulation

| | | Ovulated | | | Zygotes |
|---|---|---|---|---|---|
| | | Goats | | | (fertili- |
| | Total | (ovulation | Ovulation Point | Recovered | zation |
| | Goats | rate) | (ovulation point) | Oocytes | rate) |
| FSH | 44 | 16 | 127 | 70 | 31 |
| Group | | (36.4%)^{a} | (7.9) | (55.1) | (44.3) |
| FSH + hCG | 36 | 36 | 309 | 267 | 126 |
| Group | | (100%)^{b} | (8.6) | (86.4) | (47.2) |

^{a,b}being statically significant (P < 0.05)

As can be seen from Table I, the ovulation rate of the FSH+hCG group (100%) is higher than the FSH group (36.4%), which suggests that the combination of FSH and hCG is effective in the induction of ovulation. On the other hand, the similar fertilization rate observed for both the FSH+hCG and FSH groups, suggests that hCG does not harm the fertilization. Therefore, hCG may be advantageously used in enhancing the ovulation rate without preventing the fertilization.

In contrast to the report that FSH alone is effective in inducing the ovulation of other species of goats (Selgrath J. P. et al., Theriogenology, 34, 1195-1205 (1990); Ebert, K. M. et al., Bio/Technology, 12, 699-702 (1994); and Gootwine, E. et al., Theriogenology, 48, 485-499), the ovulation rate of the Korean native goat induced by FSH was only 36.4%. The enhanced ovulation by combining hCG with FSH observed for the Korean native goat may be attributed to the inherent physiological characteristic of the Korean native goat.

(2) Determination of the Optimal Recovery Time of the Zygote When hCG is Administered Together with FSH

In order to determine the optimal recovery time of the 1-cell stage zygote having pronuclei which is suitable in the microinjection, the procedure of Example 2 was repeated except that the zygote recovery time after the removal of the implant was varied from 62˜68, to 70˜76, and to 78˜84 hours. The developmental stage of the zygote was observed under a dissecting microscope. Results are shown in Table II.

TABLE II
Developmental stage of the zygote in various recovery times when the
combination of FSH and hCG is administered

| | | Zygotes | Developmental stage of Zygotes | | | |
|---|---|---|---|---|---|---|
| Recovery | | (fertili- | (%) | | | |
| time^{c} | Recovered | zation | 1-cell | 2-cell | 4-cell | ≧8-cell |
| (hours) | oocytes | rate) | stage | stage | stage | stage |
| 62-68 | 10 | 3 | 3 | — | — | — |
| | | (30.0) | (100) | | | |
| 70-76 | 183 | 126 | 106 | 17 | 3 | — |
| | | (68.9) | (84.1) | (13.5) | (2.4) | |
| 78-84 | 17 | 14 | 8 | 4 | 2 | — |
| | | (82.4) | (57.1) | (28.6) | (14.3) | |

^{c}time lapsing after the removal of the implant

As can be seen from Table II, the zygotes recovered at 62 to 68 hours are at the 1-cell stage and the fertilization rate is 30%. In case of the zygotes recovered at 70 to 76, the fertilization rate was much higher (70%), although some 2- and 4-cell stage zygotes were formed. The zygotes recovered at 70 to 76 hours had a much reduced content of 1-cell stage zygotes.

Therefore, to obtain 1-cell stage zygotes which is suitable in the microinjection, it is desirable to recover the zygotes at 70 to 76 hours after the removal of the implant.

### EXAMPLE 3

### Microinjection of Expression Cassette into Goat Zygote

The zygotes obtained in Example 2 were centrifuged at 12,000 rpm for 7 min. to visualize the pronuclei of each 1-cell stage zygote. Under DIC inverted microscope (Leitz, Germany) equipped with micromanipulator (Leitz, Germany), 1 to 2 pl of a DNA solution containing 4 μg/ml of expression cassette pGbc-hGCSF obtained in Example 1 was microinjected into the male pronucleus of 1-cell stage zygotes. To minimize the pH change in the course of microinjection, TL-HEPES medium (Hagen, D. R., J. Anim. Sci., 69, 1147-1150 (1991)) was used. The microinjected zygotes were cultured in m-SOF medium at 37° C. under 5% CO₂ until the transplantation. The 1-cell stage zygotes which survived the above treatment were selected.

This zygote was designated Capra hircus aegagrus embryos/pGbc-bGCSF and was deposited on Dec. 28, 1999 with the Korean Collection for Type Cultures (KCTC) (Address: Korea Research Institute of Bioscience and Biotechnology (KRIBB), #52, Oun-dong, Yusong-ku, Taejon, 305-333, Republic of Korea) under the accession number, KCTC 0718BP.

### EXAMPLE 4

### Surgical Transplantation of Microinjected Zygotes into Recipient Goat

1 to 3-year-old recipient goats (Capra hircus aegagrus) in the spontaneous estrus phase were fasted for 24 hours and then the abdominal median line of each recipient goat was locally anesthetized and cut to remove the ovary, oviduct and uterus. The 1- to 4-cell stage zygotes obtained in Example 3 were transplanted through the oviductal infundibulum using sterilized catheter, polyethylene tube having the inside diameter of 0.5 mm, outside diameter of 0.8 mm and length of 20 cm. The zygote number per recipient goat ranged from two to four. At day 30 after the transplantation, pregnancy of the recipient goats were identified with an ultrasonic diagnostic equipment (Sonorex, Medison, Korea) at day 30 after the transplantation. The zygotes were allowed to develop to term to obtain 25 progeny goats.

### TEST EXAMPLE 2

### Comparison of Spontaneous Estrus and Hormone-Induced Estrus Recipients in Terms of Pregnancy Rate and Progeny Production Rate

The microinjected zygotes obtained in Example 3 were transplanted into recipient goats respectively in spontaneous estrus and hormone-induced estrus, by repeating the procedure of Example 4. The hormone-induced recipient goats were prepared by repeating the synchronization procedure of Example 2 followed by injecting intramuscularly a dose of 400 to 600 IU of PMSF according to the response degree of the goat to the hormone at 48 hours after the removal of the implant. The pregnancy rate and progeny production rate thereof were examined. Results are shown in Table III.

TABLE III
Pregnancy rate and progeny production rate of microinjected zygotes after
transplantation

| | | | Average | Pregnant | |
|---|---|---|---|---|---|
| | | Average | Trans- | Recipient | |
| | | Ovulation | planted | (Pregnancy | |
| | Recipients | Point | Zygotes | Rate) | Progeny |
| Hormone- | 35 | 5.3 | 2.7 | 9 | 10 |
| induced | | | | (25.7) | |
| Estrus group | | | | | |
| Spontaneous | 36 | 1.8 | 2.6 | 14 | 15 |
| Estrus group | | | | (38.9) | |

As can be seen from Table III, the average ovulation point of the spontaneous estrus group is lower than that of hormone-treating group while the pregnancy rate of spontaneous estrus group is higher than that of hormone-treating group.

### EXAMPLE 5

### Identification of Transgenic Goat

(1) Isolation of Genome DNA

From 10 to 30-day-old progeny goats obtained in Example 4, a portion of the ear tissue of each goat was cut in a size of 0.5 cm and then transferred to a 15 ml test tube, and 4 ml of a lysis solution(10 mM Tris-Cl (pH 8.0), 0.1 mM EDTA and 0.5% SDS) was added thereto. The resulting mixture was kept at 55° C. for 16 hours to lyse the tissue.

The lysed tissue cells were subjected to phenol extraction and ethanol precipitation according to Sambrook, J. et al. (Molecular cloning: a laboratory manual, 2nd ed. Cold Spring Harbor Laboratory Press, New York (1989)) to obtain a pure genome DNA. The purified DNA was dissolved in distilled water to a final concentration of 0.5 μg/μl.

(2) PCR

1 μl of each progeny's genome DNA obtained in (1) was subjected to PCR using primers GB2(SEQ ID NO: 4) and GCSF2 (SEQ ID NO: 5) or primers GB2 (SEQ ID NO: 4) and GCSF3 (SEQ ID NO: 6). The PCR was carried out by incubating at 94° C. for 4 min. to denature DNA; and repeating the thermal cycle 30 times, each cycle being composed of: 94° C. for 1 min., 55° C. for 1 min. and 72° C. for 1 min. The PCR product thus obtained was subjected to electrophoresis in 6% polyacrylamide sequencing gel, followed by autoradiography. The primer GB2 (SEQ ID NO: 4) has the nucleotide sequence of the 5′-portion of sense strand of β-casein gene (the region ranging from 1621st to 1640th nucleotides) and primers GCSF2 (SEQ ID NO: 5) and GSF3 (SEQ ID NO: 6) have the nucleotide sequences complementary to the 3′-portions of sense strand of hG-CSF (the regions ranging from 511th to 530th and 681st to 698th nucleotides, respectively).

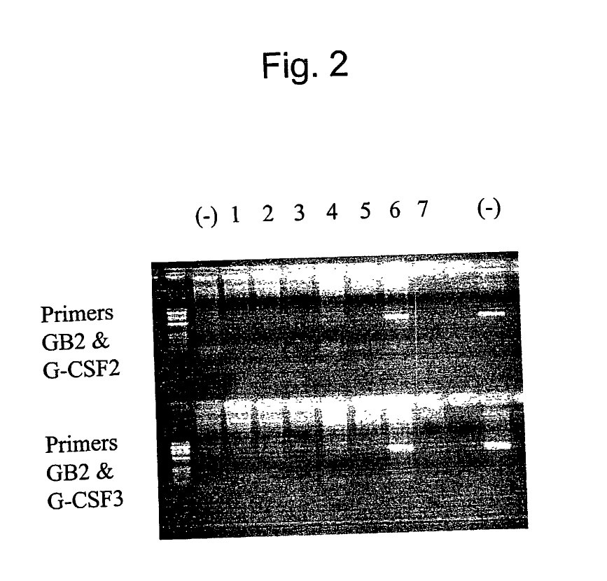

The PCR products were electrophoresed on agarose gel and the result is shown in FIG. 2, wherein lanes 1 to 7 are the PCR products of the respective progeny goats; lane (−), parental wild-type goat; and lane (+), the mixture of parental wild-type goat genome DNA and plasmid pGbc-hGCSF. On lane 6 of FIG. 2, one can observe a 480 bp band obtained by PCR using primers GB2 (SEQ ID NO: 4) and GCSF2 (SEQ ID NO: 5), and a 540 bp band obtained by PCR using primers GB2 (SEQ ID NO: 4) and GCSF3 (SEQ ID NO: 6). This confirms that the progeny of lane 6 is a transgenic goat introduced with expression cassette pGbc-hGCSF.

(3) Southern Blot Analysis

10 μg of the progeny genome DNA obtained in (1) was cleaved with HindIII and the resulting fragments were electrophoresed on 0.8% agarose gel and transferred to a nylon membrane according to Sambrook, J. et al. (vide supra). The DNA-adsorbed nylon membrane was pre-hybridized with a pre-hybridization solution(6× SSC, 5× Denhardt's reagent and 0.5% SDS) at 68° C. for 2 hours and then hybridized with a ³²P-labelled hG-CSF probe prepared by randomly priming the HindIII/NaeI fragment of hG-CSF gene using [α-³²P] dCTP.

After completion of the reaction, the nylon membrane was sequentially washed with 2× SSC/0.1% SDS solution at room temperature, with the identical solution at 65° C. for 10 min., and with 1× SSC/0.1% SDS solution at 65° C. for 10 min. An X-ray film was laid on the nylon membrane, exposed at −70° C. for three days and then developed.

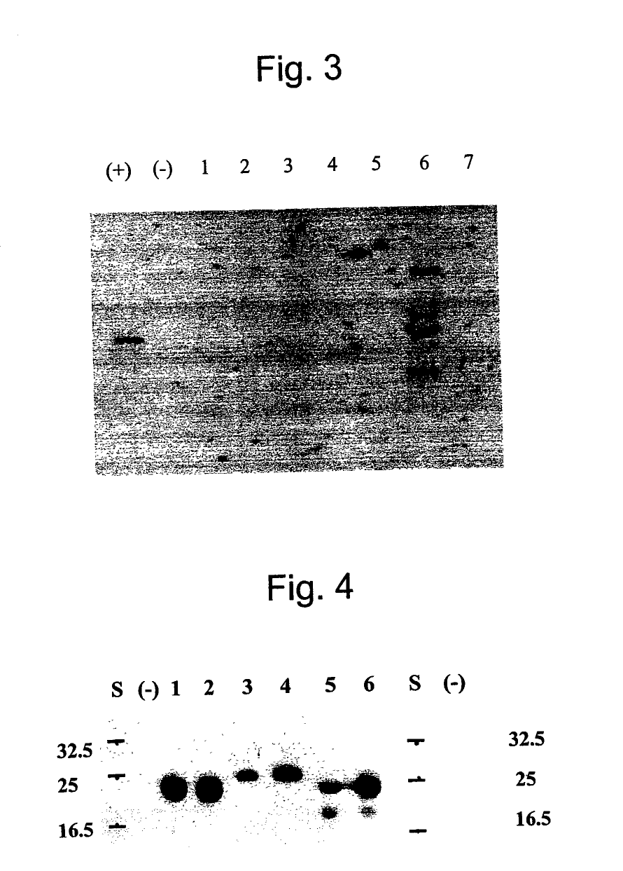

The results are shown in FIG. 3 wherein lanes 1 to 7 represents the genome DNAs of the respective progeny goats; lane (−), genome DNA of parental wild-type goat; and lane (+), the mixture of parental wild-type goat genome DNA and plasmid pGbc-hGCSF. The results in FIG. 3 suggest that the progeny of lane 6 is a transgenic goat introduced with expression cassette pGbc-hGCSF.

By repeating the above procedure, two transgenic goats were identified among a total of 25 progeny goats.

### EXAMPLE 6

### Western Blot Analysis of hG-CSF Contained in Milk of Transgenic Goat

After bringing the transgenic goats obtained in Example 5 to bear offsprings, the milk at day 2 (colostrum) and 5 were taken. To the milk, an equal volume of 1× PBS was added and the resulting mixture was kept at 4° C. for 1 hour and then centrifuged at 13,000 rpm for 15 min. to obtain a supernatant(milk-serum). 2 μl of the supernatant was subjected to 15% SDS-PAGE. The procedure was repeated using commercial rHuG-CSF derived from E. coli (Kirin, Japan) and rHuG-CSF derived from CHO cells (Jugai, Japan), respectively, as comparative groups, and a milk-serum of the parental wild-type goat. The proteins separated on the gel were transferred on a nitrocellulose membrane (Amersham pharmacia biotech, USA) according to the method well known in the art (Protein Methods, Daniel M bollag and Stuart J. Edelstein, Wiley-Liss, 1991). The membrane was treated with a blocking solution (1× PBS containing 3% of skim milk) for 1 hour in a shaker. The membrane was treated for 1 hour with a solution prepared by diluting anti-hG-CSF mouse IgG (R&D systems, USA) 1,000-fold with 10 ml of the blocking solution and further diluted three-fold with 300 ml of 1× PBS for 5 min. in a shaker. The membrane was treated for 1 hour with 10 ml of a solution prepared by diluting horseradish peroxidase-conjugated anti-mouse IgG antibody 1,000-fold with the blocking solution. The membrane was treated three times with 1× PBS for 5 min and developed using ECL kit (Amersham pharmacia biotech, USA).

The results are shown in FIG. 4, wherein lane 1 is 50 ng of rHuG-CSF; lane 2, 100 ng of E. coli rHuG-CSF; lane 3, 50 ng of CHO rHuG-CSF; lane 4, 100 ng of CHO rHuG-CSF; lane 5, 1 μl of the milk-serum of the transgenic goat at day 2; lane 6, 1 μl of the milk-serum of the transgenic goat at day 5; lane S, protein molecular weight markers; and lane (−), the milk-serum of the parental wild-type goat. As can be seen from FIG. 4, a band of about 18 kDa protein is present in the milk of the transgenic goat, which is identical with the commercial hG-CSFs. Further, the band density of the milk-serum at day 5 is stronger than that of the milk-serum at day 2. This suggests that the hG-CSF concentration in the milk-serum ranges from 50 to 100 μg/ml. Thus, the transgenic goat releases a large quantity of hG-CSF in milk.

### EXAMPLE 7

### hG-CSF Concentration in the Milk of Transgenic Goat

To determine the hG-CSF concentration in the milk of the transgenic goat, the milk of the transgenic goat obtained in Example 6 was diluted 4-fold with a buffer solution (20 mM Trizma base pH 7.4 and 1 mM EDTA), and centrifuged three times with 27,000× g at 4° C. for 20 min. to remove lipids and saccharides. The hG-CSF concentration in the supernatant (milk-serum) was determined using the commercial granulocyte-colony stimulating factor (G-CSF) ELISA (Cat # DCS50, R&D systems, USA).

### EXAMPLE 8

### Activity of hG-CSF Contained in Milk of Transgenic Goat

HL-60 cells originated from human bone marrow (ATCC CCL-240) were cultured in RPMI 1640 medium containing 10% fetal bovine serum at 37° C. under 5% CO₂ condition. The number of cells was adjusted to 2.2×10⁵ cell/ml and DMSO was added to a final concentration of 1.25%(v/v). 90 μl of the cell culture(about 2×10⁴ cells) was added to each well of a low evaporation 96 well-plate (NUNC, Denmark) and cultured at 37° C. under 5% CO₂ condition for 48 hours.

Each of the milk-serum of the transgenic goat obtained in Example 6, the milk-serum of the parental wild-type goat and the commercial hG-CSF (Choongwae Pharma Corporation) was diluted to a final hG-CSF concentration of 500 ng/ml with RPMI 1640 medium, and subjected to sequential 2-fold dilution with RPMI 1640 medium.

2 μg of the commercial hG-CSF(Choongwae Pharma Corporation) was added to 10 ml of the milk-serum of the parental wild-type goat to obtain a mixture and 10 μl of the mixture was added to a well containing HL-60 cells and cultured at 37° C. for 48 hours.

To examine the proliferation degree of the cells in the culture, each culture was treated with CelITiter96™ (cat# G4100, Promega, USA) and the optical density thereof was measured at a wavelength of 670 nm.

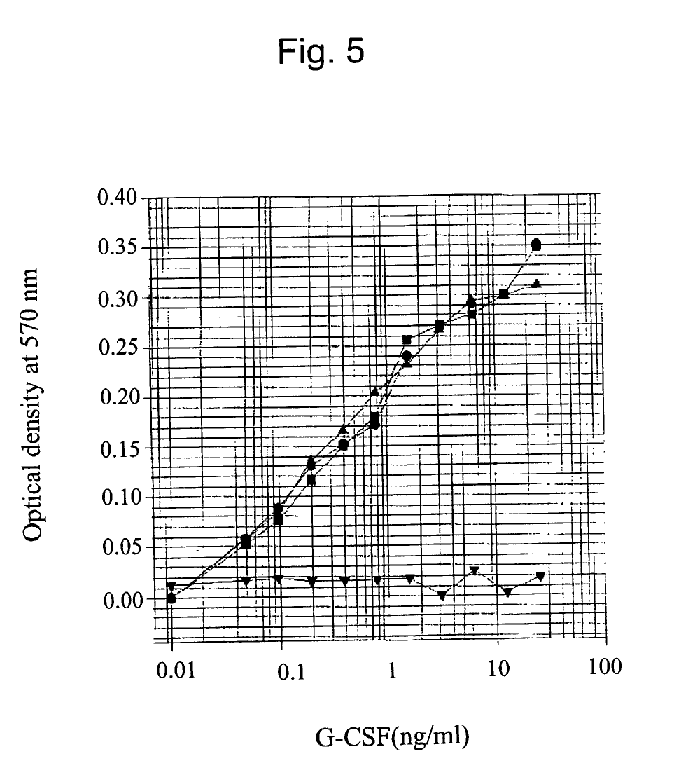

The results are shown in FIG. 5, wherein -&Ovalsolid;- represents the commercial hG-CSF; -&Rectversolid;-, the mixture of the commercial hG-CSF and the milk-serum of the parental wild-type goat; -▴-, the milk-serum of the transgenic goat; and -▾-, the milk-serum of the transgenic goat. As can be seen from FIG. 5, the milk-serum of the transgenic goat has a cell-proliferating activity identical with the commercial hG-CSF, while that of the parental wild-type goat does not influence the cell proliferation. Further, the proliferation of the HL-60 cells is induced by hG-CSF contained the milk-serum of the transgenic goat which is equivalent to that of the known hG-CSF.

While the subject invention has been described and illustrated with reference to the preferred embodiments only, it may be apparent to those skilled in the art that various changes and modifications can be made therein without departing from the spirit and scope of the present invention which is defined in the appended claims.

What is claimed is:

1. A goat zygote which comprises a nucleic acid construct containing a nucleotide sequence of a goat β-casein promoter and a nucleic acid sequence encoding human granulocyte-colony stimulating factor (hG-CSF).

2. The goat zygote of claim 1, wherein the goat is Capra hircus aegagrus.

3. The goat zygote of claim 1, wherein the nucleic acid construct is expression vector pGb-hGCSF (SEQ ID NO: 1).

4. The goat zygote of claim 3 which is Capra hircus aegagrus embryos/pGbc-bGCSF (KCTC 0718BP).

5. A method for preparing the goat zygote of claim 1 which comprises microinjecting a nucleic acid construct containing a nucleotide sequence of a goat β-casein promoter and a nucleotide sequence encoding hG-CSF into an intact goat zygote.

6. A method for preparing an intact goat zygote which comprises synchronizing a female goat, superovulating the female goat, mating the superovulated female goat with a male goat and recovering a zygote from the mated female goat, characterized in that the synchronizing step is conducted by administering norgestomet and estradiol to the female goat, inserting an implant containing norgestomet to the female goat and removing the implant; and the superovulating step is conducted by administering to the female goat, sequentially at predetermined time intervals, a combined dose of pregnant mare serum gonadotropin(PMSG) and follicle stimulating hormone(FSH), divided doses of FSH, and a combined dose of FSH and human chorionic gonadotropin(hCG).

7. The method of claim 6, wherein the goat is Capra hircus aegagrus; and in the synchronizing step, the administration of PMSG and FSH is carried out at 60 hours before the removal of the implant, the FSH administration is carried out six times every 12 hours after the administration of PMSG and FSH, and the administration of FSH and hCG is carried out 12 hours thereafter.

8. A transgenic goat producing milk containing hG-CSF, which is developed from the goat zygote of any one of claims 1 to 4.

9. A process for preparing the transgenic goat of claim 8 which comprises transferring the goat zygote of any one of claims 1 to 4 into a female goat and allowing the goat zygote to develop to term.

10. A method for producing hG-CSF which comprises producing milk from the transgenic goat of claim 8 and recovering the hG-CSF from the milk.

11. A milk composition comprising hG-CSF, which is produced from the transgenic goat of claim 8.

12. hG-CSF which is produced by method of claim 10.

13. A pharmaceutical composition which comprises the hG-CSF of claim 12 and a pharmaceutically acceptable carrier.


SEQUENCE LISTING (6 sequences)

SEQ ID NO: 1 - DNA, 3686 bp, Artificial Sequence
  expression cassette pGbc-hGCSF
gagctctttagtatattgttaaggatttcttgatcaagattttacctacttttctggtccaattggtgagagacagtcataaggaaatgctgtgtttattgcacaatatgtaaagcatcttcctgagaaaataaaagggaaatgttgaatgggaaggatatgctttcttttgtattccttttctgagaaatcagactttttcacctgtggccttggcacaaaagctaacaaataaaggcatatgaagtagccaaggccttttctagtatatctatgacactgagttcatttcatcatttattttcctgacttcctcctgggtccatatgagcagtcttagaatgaatattagctgaataatccaaatacatagtagatgttgatttgggttttctaagcaatccaagacttgtatgacagtaagatgtattaccatccaacaacacacatctcagcatgatataaatgcaaggtatattgtgaagaaaaatttttaattatgtcaaagtgcttactttagaaggtcatctatctgtcccaaagctgtgaatatatatattgaaggtaatgaatagatgaagctaaccttgtaaaaatgagtagtgtgaatacaactacaattatgaacatctgtcactaaagaggcaaagaaacttgaagattgcttttgcaaatgggctcctattaataaaaagtacttttgaggtctggctcagactctattgtagtacttagggtaataccctcctcctgtatgggctttcattttctttcttgcttccctcatttgcccttccatgaatgactagctgataaagcattgactataaaagatatgaggccaaacttgagctgtcccattttaataaatctgtataataatattgttctacaaaagtattatctaaataaatgttactttctgtcttaaaatccctcaacaaatccccactatctagaggatccgattgacattccctggaatcacagcatgctttgtctgccattatctgacccctttctctttctctcttctcacctccatctactcctttttccttgcaattcatgacccagattcactgtttgatttggcttgcatgtgtgtgtgctgagttgcgtctgactgttatcaaccccatgaatgatagtccaccaggctctactgtccatgaaattttccagtcaagaatactggagtggattgcatttcctactccatttgattaatttagtgacttttaaatttctttttccatattcgggagcctattcttcctttttagtctatactctcttcactcttcaggtctaaggtatcatcgtgtgcttgttagcttgttactttctccattatagcttaagcactaacaactgttcaggttggcatgaaattgtgttctttgtgtggcctgtatatttctgttgtgtattagaatttaccccaagatctcaaagacccactgaatactaaagagacctcattgtggttacaataatttggggactgggccaaaacttccgtgcatcccagccaagatctgtagctactggacaatttcatttcctttatcagattgtgagttattcctgttaaaatgctccccagaatttctggggacagaaaaataggaagaattcatttcctaatcatgcagatttctaggaattcaaatccactgttggttttatttcaaaccacaaaattagcatgccattaaatactatatataaacagccactaaatcagatcattatccattaagcttgatatcgaattcctgcagcccagccccacccagacccatggctggacctgccacccagagccccatgaagctgatgggtgagtgtcttggcccaggatgggagagccgcctgccctggcatgggagggaggctggtgtgacagaggggctggggatccccgttctgggaatggggattaaaggcacccagtgtccccgagagggcctcaggtggtagggaacagcatgtctcctgagcccgctctgtccccagccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgaggtgggtgagagggctgtggagggaagcccggtggggagagctaagggggatggaactgcagggccaacatcctctggaagggacatgggagaatattaggagcagtggagctggggaaggctgggaagggacttggggaggaggaccttggtggggacagtgctcgggagggctggctgggatgggagtggaggcatcacattcaggagaaagggcaagggcccctgtgagatcagagagtgggggtgcagggcagagaggaactgaacagcctggcaggacatggagggaggggaaagaccagagagtcggggaggacccgggaaggagcggcgacccggccacggcgagtctcactcagcatccttccatccccagtgtgccacctacaagctgtgccaccccgaggagctggtgctgctcggacactctctgggcatcccctgggctcccctgagcagctgccccagccaggccctgcagctggtgagtgtcaggaaaggataaggctaatgaggagggggaaggagaggaggaacacccatgggctcccccatgtctccaggttccaagctgggggcctgacgtatctcaggcagcaccccctaactcttccgctctgtctcacaggcaggctgcttgagccaactccatagcggccttttcctctaccaggggctcctgcaggccctggaagggatctcccccgagttgggtcccaccttggacacactgcagctggacgtcgccgactttgccaccaccatctggcagcaggtgagccttgttgggcagggtggccaaggtcgtgctggcattctgggcaccacagccgggcctgtgtatgggccctgtccatgctgtcagcccccagcatttcctcatttgtaataacgcccactcagaagggcccaaccactgatcacagctttcccccacagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctgagccaagccctccccatcccatgtatttatctctatttaatatttatgtctatttaagcctcatatttaaagacagggaagagcagaacggagtctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatccccgggtacc

SEQ ID NO: 2 - DNA, 29 bp, Artificial Sequence
  primer CAS-F1
tgatcgcgagtccaccaggctctactgtc

SEQ ID NO: 3 - DNA, 26 bp, Artificial Sequence
  primer CAS-R1
gagaagcttaatggataatgatctga

SEQ ID NO: 4 - DNA, 20 bp, Artificial Sequence
  primer GB2
tggggacagaaaaataggaa

SEQ ID NO: 5 - DNA, 20 bp, Artificial Sequence
  primer GCSF2
atcttcctcacttgctttaa

SEQ ID NO: 6 - DNA, 18 bp, Artificial Sequence
  primer GCSF3
ctctcacccacctcactc